(19) United States
(12) Patent Application Publication
(10) Pub. No.: US 2013/0116653 A1
(43) Pub. Date: May 9, 2013

(54) INTRAVENOUS INFUSION DEVICE WITH DRIP CHAMBER AND ELASTIC FLOAT

(71) Applicants: Ian Guy, London (GB); Graham Bell, Renfrewshire (GB); James Limb, North Yorkshire (GB)

(72) Inventors: Ian Guy, London (GB); Graham Bell, Renfrewshire (GB); James Limb, North Yorkshire (GB)

(21) Appl. No.: 13/701,766

(22) PCT Filed: Jun. 3, 2011
(86) PCT No.: PCT/GB2011/051054

§ 371 Date: Jan. 14, 2013

(30) Foreign Application Priority Data
Jun. 4, 2010 (GB) 1009375.5

(51) Int. Cl. A61M 5/14

(52) U.S. Cl. CPC A61M 5/1411
USPC 604/507; 604/254

(57) ABSTRACT

An intravenous infusion device (10) comprises a fluid drip chamber (11) having an inlet (12) and an outlet (13). A floating plug (14) provides an indication of fluid level and when the fluid is exhausted the plug (14) settles in the outlet (13) and, being made from a soft deformable material, effectively forms a bung preventing air entering the line (4). It is thus a simple matter to replace an associated fluid bottle (2) and allow the replenished fluid to flow with minimal interruption or intervention. By providing the plug (14) with a flexible skirt or fin (15) and providing the chamber (11) with a conical end section (16) an enhanced seal is provided by the fin (15) engaging the conical section (16). In a further enhancement the conical section (16) has an opaque lower section (17) and the plug (14) is thus obscured from view as the fluid is depleted providing an enhanced visual indication that the fluid bottle (2) is empty. The plug (14) may advantageously also be coloured at least partially green such that the presence of a green indicator indicates that the infusion device is functioning.

This invention relates to an intravenous infusion device and in particular to an air free administration device for intravenous infusions.

### BACKGROUND

Intravenous drips are used widely in medical environments as a means of providing a steady measured supply of drugs and fluids to a patient. In many instances the fluid supply is provided by a self collapsing plastics material bag; external air pressure collapsing the bag and maintaining fluid flow as the contents are depleted. Atmospheric air cannot normally enter the system in use. However many drugs cannot be supplied this way because they are more reactive and as collapsible plastics material bags are to some degree porous there is thus a tendency for the contents to “go off”. The usual solution to this problem is to supply such drugs in rigid glass bottles. In order for infusion to take place with a rigid container air pressure must act directly on the contents and this happens by allowing air to enter the bottle. When the solution comes to an end this can leave air entrained in the line—that is air bubbles can be drawn into the infusion line. Air entrainment is at best of nuisance value to nurses and anaesthetists requiring removal time which temporarily halts a patients drug supply and, in theatre, may distract the anaesthetist from other duties possibly leading to the need to pause a surgical procedure which is less than ideal.

More seriously unnoticed and undealt with entrainments can be infused into the patient's bloodstream which carries a risk of causing a fatal air embolism.

Previously solutions to this problem have been proposed.

In some existing drip chambers a rigid floating solid ball is provided. This provides a visual indication of fluid level in the chamber. However when the chamber empties and the ball settles in the chamber outlet it does not provide an effective seal thus allowing air to enter the line. Volumetric infusion pumps can be used. Such pumps have an alarm system to detect air in the lines. However infusion pumps can cost from hundreds to thousands of pounds and thus from an economic perspective it is not practical for hospitals and healthcare bodies to purchase an infusion pump for every drip therefore inevitably leaving some patients at risk. In addition such pumps, even when present, are only a partial solution to the problem in that they do not prevent air entrainment—they only stop infusion when air is detected in a drip line. Consequently medical staff still need to spend time removing air from lines and there is therefore still a degree of staff distraction.

Another proposal is the intrafix safeset produced by Braun. This equipment is specifically designed to prevent air entrainment from rigid containers. It works by employing a very fine hydrophilic membrane filter which, when wet, creates a high surface tension preventing air from passing through into the drip line. There are however a number of shortcomings with this product making its use limited. In particular the product can only be used with crystalloid solutions and not colloid solutions or blood products—which make up a significant proportion of the fluids which are normally infused. If the equipment was used with such colloid solutions or blood products it would simply filter out the suspended components of the fluid—which is the part you actually want to administer to the patient. Hence this product leaves a group of patients at risk as they cannot be protected by it. A further disadvantage is that the product can only be used with a compatible Braun infusion pump. There is therefore a compatibility issue which introduces the potential for human error if used with the wrong type of pump or the need for expensive upgrade of equipment to use only compatible pumps.

There is therefore a clearly demonstrated need for a simple universally applicable preventive product which can be used in all medical applications requiring a drip for a patient.

### BRIEF SUMMARY OF THE DISCLOSURE

In accordance with the present inventions there is provided an intravenous infusion device comprising a drip chamber having an inlet for receiving intravenous fluid from a supply container and an outlet for supplying said fluid to a patient infusion line characterised in that an elastic material float is provided having a formation complimentary to the chamber outlet such that it sealably engages therewith on the emptying of fluid from the chamber.

Preferably the float has a circumferentially arranged flexible fin which provides a low pressure seal around the chamber exit on engagement therewith.

Preferably also the float is generally conical in profile and engages with a complimentary profile at the chamber exit.

Advantageously the chamber has an upper transparent section and a lower opaque section arranged such that the float is obscured from view when the chamber is empty.

More advantageously the float may have a coloured indicator section, preferably green in colour, visible when sufficient fluid remains in the chamber.

### BRIEF DESCRIPTION OF THE DRAWINGS

Embodiments of the invention are further described hereinafter with reference to the accompanying drawings, in which:

FIG. 1 is a schematic view of prior art intravenous infusion apparatus illustrating normal fluid operation;

FIG. 2 is a view corresponding to FIG. 1 illustrating a fluid empty condition.

FIG. 3 is a side view of one embodiment of an intravenous infusion device of the present invention containing intravenous fluid;

FIG. 4 is a side view of the intravenous infusion device of FIG. 1 empty of intravenous fluid;

FIG. 5 is an exploded side view of an intravenous infusion device of the present invention;

FIG. 6 is a schematic view of an intravenous infusion device of the present invention containing intravenous fluid and incorporating a status indicator; and

FIG. 7 is a schematic view of an intravenous infusion device of the present invention empty of fluid and incorporating a status indicator.

### DETAILED DESCRIPTION

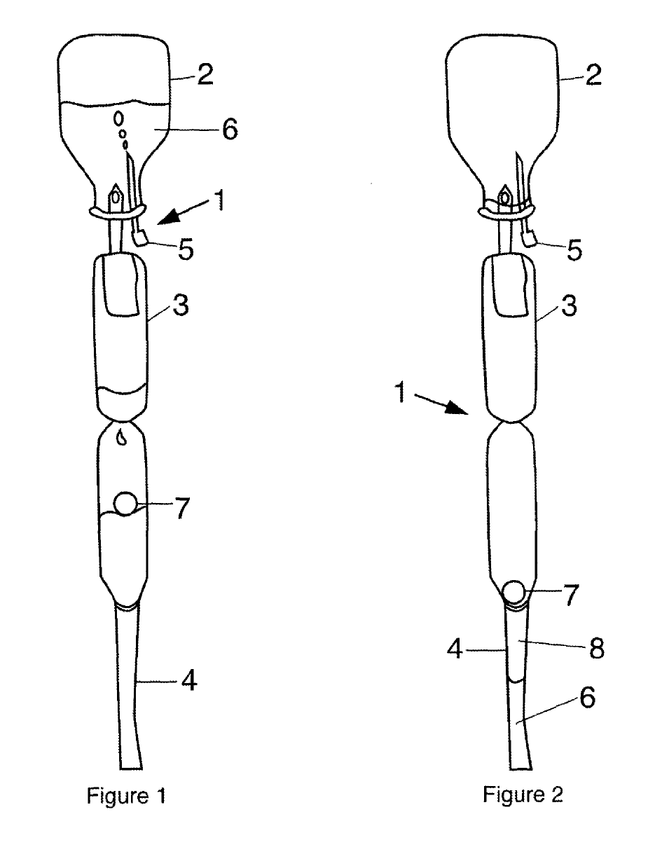

Referring to FIG. 1 conventional intravenous infusion apparatus is shown generally at 1 and comprises a solution glass bottle 2 which is connected to a drip chamber 3 which in turn leads to an infusion line 4 which connects to a patient. The bottle 2 has an air bleed 5 which allows an IV solution 6 to run through the drip chamber 3 to the line 4 and hence the patient. An indicator float ball 7 provides a visual indication of the fluid level in the chamber 3. A drawback of such apparatus is that when the fluid supply is exhausted an air bubble 8 can be created in the line 4 as shown in FIG. 2. Thus when a new fluid bottle 2 is attached by a medical operative extra time and skill is required to ensure that the air bubble 8 is removed from the line 4 and cannot travel down to enter the patient which may have dangerous implications.

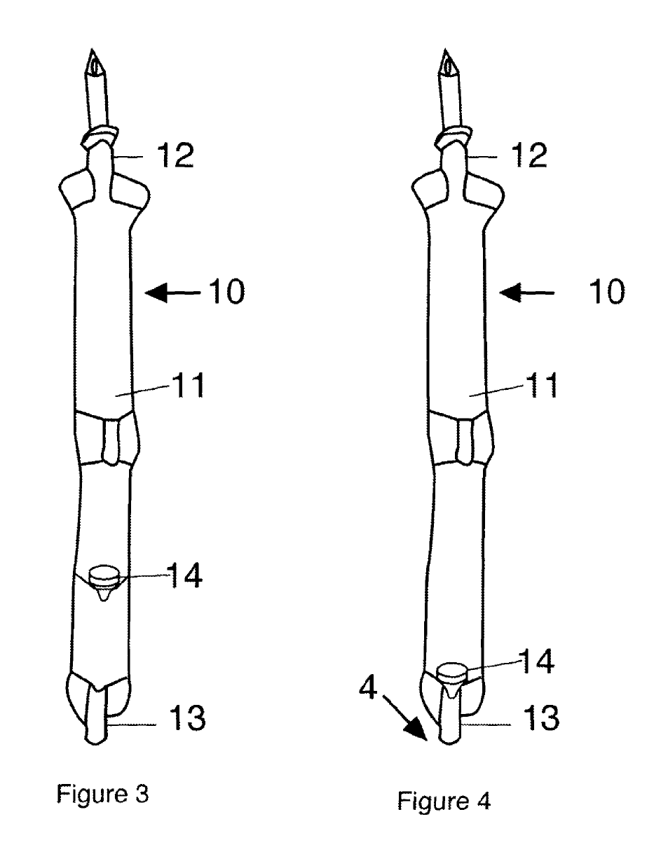

Turning now to FIGS. 3 & 4 an intravenous infusion device of the present invention is shown generally at 10 and comprises a fluid drip chamber 11 having an inlet 12 and an outlet 13. A generally conically shaped floating plug 14 made from a soft deformable material provides an indication of fluid level, as with the prior art, but when the fluid is exhausted it settles in the outlet 13 effectively forming a bung and preventing air entering the line 4. It is thus a simple matter to replace the bottle 2 and allow the replenished fluid to flow with minimal interruption or intervention from the medical operative. Not only does this provide a time saving but also the potential for errors to occur is reduced.

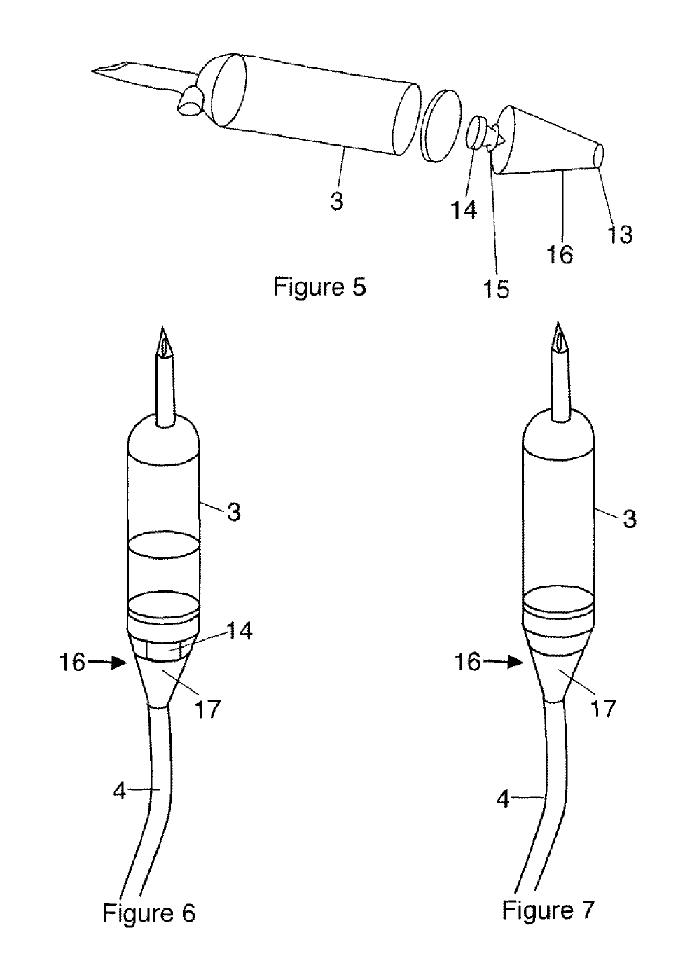

Turning now to FIG. 5 further details of the basic concept are illustrated. It will be appreciated that this type of infusion apparatus is particularly simple and is effectively gravity fed. Consequently it is a low pressure environment which presents certain challenges in ensuring that the plug 14 provides an air tight seal. This is partly ensured by forming the plug 14 from an injection moulded soft deformable plastics material which readily co-operates with the outlet 13 of the chamber 3. However by providing the plug 14 with a flexible skirt or fin 15 and providing the chamber with a conical end section 16 an improved seal is provided by the fin 15 engaging the conical section 16.

Referring to FIGS. 6 & 7 a further refinement is illustrated. In this embodiment the conical section 16 has an opaque lower section 17 and the plug 14 is thus obscured from view as the fluid is depleted providing an enhanced visual indication that the fluid bottle 2 is empty. The plug 14 may advantageously also be coloured at least partially green such that at a quick glance the presence of a green indicator indicates to a medical operative that the infusion device is functioning and no action is required. Alternatively if the green indicator plug is not visible this indicates that infusion has ceased and action is therefore required.

The infusion device as described provides several advantages. Operation is automatic; no setup is required and an empty bottle can simply be replaced by a full one and infusion restarted without any additional operations required. In addition it is suitable for use with any common infusion fluid reducing the possibility for human error.

The device can be a straight replacement for existing equipment and is compatible with other sections of existing equipment making its introduction and use simple and straightforward without the need to replace or overhaul other existing equipment.

Clear and unambiguous indication of infusion fluid level is provided such that, when used in theatre, anaesthetists are readily aware of the remaining fluid level so that delays caused by fluid replenishment can be minimised.

The removal of the need for medical staff to spend time removing air from lines increases staff productivity.

Throughout the description and claims of this specification, the words “comprise” and “contain” and variations of them mean “including but not limited to”, and they are not intended to (and do not) exclude other components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers and characteristics described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. An intravenous infusion device comprising a drip chamber having an inlet for receiving intravenous fluid from a supply container and an outlet for supplying said fluid to a patient infusion line wherein an elastic material float is provided having a formation complimentary to the chamber outlet such that it sealably engages therewith on the emptying of fluid from the chamber.

2. An intravenous infusion device as claimed in claim 1 wherein the float has a circumferentially arranged flexible fin which provides a low pressure seal around the chamber exit on engagement therewith.

3. An intravenous infusion device as claimed in claim 1 wherein the float is generally conical in profile and engages with a complimentary profile at the chamber exit.

4. An intravenous infusion device as claimed claim 1 wherein the chamber has an upper transparent section and a lower opaque section arranged such that the float is obscured from view when the chamber is empty.

5. An intravenous infusion device as claimed in claim 4 wherein the float has a colored indicator section visible when sufficient fluid remains in the chamber.

6. A method of intravenous infusion using a device of claim 1, comprising the steps of:
a) providing a patient with an infusion line,
b) attaching the infusion device of claim 1 to the infusion line, wherein the infusion device comprises a solution to be administered to the patient via the infusion line, and
c) administering the solution to the patient using the infusion device.

7. An intravenous infusion device as claimed in claim 2 wherein the float is generally conical in profile and engages with a complimentary profile at the chamber exit.

8. An intravenous infusion device as claimed in claim 2 wherein the chamber has an upper transparent section and a lower opaque section arranged such that the float is obscured from view when the chamber is empty.

9. An intravenous infusion device as claimed in claim 3 wherein the chamber has an upper transparent section and a lower opaque section arranged such that the float is obscured from view when the chamber is empty.

10. An intravenous infusion device as claimed in claim 8 wherein the float has a colored indicator section visible when sufficient fluid remains in the chamber.

11. An intravenous infusion device as claimed in claim 9 wherein the float has a colored indicator section visible when sufficient fluid remains in the chamber.

12. The method of claim 6, wherein the float has a circumferentially arranged flexible fin which provides a low pressure seal around the chamber exit on engagement therewith.

13. The method of claim 6, wherein the float is generally conical in profile and engages with a complimentary profile at the chamber exit.

14. The method of claim 6, wherein the chamber has an upper transparent section and a lower opaque section arranged such that the float is obscured from view when the chamber is empty.

15. The method of claim 14, wherein the float has colored indicator section visible when sufficient fluid remains in the chamber.
